(19) United States
(12) Patent Application Publication
(10) Pub. No.: US 2025/0268995 A1
(43) Pub. Date: Aug. 28, 2025

(54) COMPOSITIONS FOR BREAKDOWN OF GLUTEN PROTEINS

(71) Applicants: Aditi Avinash, Castle Pines, CO (US); Sujatha Venkataraman, Superior, CO (US)

(72) Inventors: Aditi Avinash, Castle Pines, CO (US); Sujatha Venkataraman, Superior, CO (US)

(21) Appl. No.: 19/060,575

(22) Filed: Feb. 21, 2025

Related U.S. Application Data
(60) Provisional application No. 63/557,216, filed on Feb. 23, 2024.

(51) Int. Cl. A61K 38/48; A61K 35/745; A61K 35/747

(52) U.S. Cl. CPC A61K 38/4873; A61K 35/745; A61K 35/747; A61K 38/488; C12Y 304/22002; C12Y 304/22014; C12Y 304/22032; C12Y 304/23001

(57) ABSTRACT

Disclosed are compositions for promoting the breakdown of gluten proteins. In some example embodiments, the composition comprises one or more of papain, bromelain, and actinidin. In some example embodiments, the composition comprises papain, bromelain, and actinidin. In some example embodiments, the ratio of papain to bromelain to actinidin is 1:2:3. In some example embodiments, the composition also comprises a digestive enzyme. In some example embodiments, the composition also comprises a probiotic.

### PRIOR RELATED APPLICATIONS

This Non-Provisional Application claims the benefit of and priority to U.S. Provisional Application No. 63/557,216, filed Feb. 23, 2024, which is hereby incorporated by reference in its entirety.

### FIELD OF THE INVENTION

Disclosed are compositions for the breakdown of gluten proteins.

### BACKGROUND

Celiac disease (CeD) is an autoimmune disease that occurs in genetically predisposed people. When a person with CeD consumes gluten, a protein present in wheat, rye, barley, and other grains, it causes an immune response that attacks the small intestine. The resulting damage to the small intestine's villi impairs nutrient absorption, and, over time, can lead to additional serious health problems. Furthermore, the consumption of gluten can cause immediate debilitating abdominal pain, nausea, and gastrointestinal distress. Currently the only option for those with CeD to avoid the immediate symptoms and long-term sequelae of CeD is to maintain a strict gluten free diet.

Previous studies have investigated the idea of promoting gluten protein breakdown upon ingestion, as the smaller amino acid breakdown products may not trigger an immune response in the small intestine of a person with CeD. However, there is still a need for safe and effective therapeutic interventions that result in the breakdown of gluten proteins.

### SUMMARY OF THE INVENTION

In some embodiments, the present disclosure provides compositions for the breakdown of gluten proteins. In some embodiments, the gluten proteins comprise gliadin. In some embodiments, the gluten proteins comprise 33-mer gliadin peptides. In some embodiments, the gluten proteins comprise 33-mer deaminated gliadin peptides.

In some embodiments, the composition comprises fruit extract-derived enzymes for the breakdown of gluten proteins. In some embodiments, the fruit extract-derived enzymes include one or more of papain, bromelain, and actinidin.

In some embodiments, the fruit extract-derived enzymes comprise papain, bromelain, and actinidin. In some embodiments, the ratio of papain to bromelain to actinidin is 1:2:3 (“1-2-3 EC mix”).

In some embodiments, the composition additionally comprises at least one digestive enzyme. In some embodiments, the digestive enzyme comprises one or more of protease, pepsin, amylase, lipase, or lactase.

In some embodiments, the composition additionally comprises a probiotic. In some embodiments, the probiotic comprises one or more strains of Lactobacillus or Bifidobacterium.

### BRIEF DESCRIPTION OF THE FIGURES

FIG. 1 depicts the immuno-dominant 33-mer gliadin peptide that initiates immune responses in CeD patients; the depicted deamidation by transglutaminase (TG2) results in the 33-mer deamidated gliadin peptide that causes T-cell stimulation in CeD patients.

FIG. 2 depicts flow charts of the protocols used to test the effectiveness of combinations of enzymes in inducing breakdown of gliadin peptides.

FIG. 3 depicts the cleaved sites of 33-mer gliadin peptides and 33-mer deamidated gliadin peptides following treatment with 1-2-3 EC mix as well as the cleaved sites of 33-mer gliadin deamidated peptides following treatment with 1/2/3 sequential monotherapy.

FIG. 4 depicts the quantitation of breakdown amino acid fragments of 33-mer gliadin peptides by 1-2-3 EC mix and 1/2/3 sequential monotherapy via LC/MS.

FIG. 5 depicts the protocol and results of testing the efficacy of 1-2-3 EC mix degradation of 33-mer gliadin peptide in an environment representative of the stomach.

FIG. 6 shows 1-2-3 EC mix significantly degraded 33-mer gliadin peptides at pH 6 and at pH 2-3 compared to 1/2/3 sequential monotherapy or a store bought pill as measured by ELISA.

FIG. 7 shows the combination of pepsin in HCl and 1-2-3 EC mix completely degrades 33-mer gliadin peptide (lane 7), visualized via SDS-PAGE gel stained with coomassie blue.

FIG. 8 shows treating 33-mer gliadin peptides with 1-2-3 EC mix and pepsin prevents T cell stimulation measured via IL-2 secretion from mouse T-cells transduced with human T-cell receptors isolated from CeD patients.

### DETAILED DESCRIPTION

### Definitions

The present disclosure now will be described more fully hereinafter. The disclosure may be embodied in many different forms and should not be construed as limited to the aspects set forth herein; rather, these aspects are provided so that this disclosure will satisfy applicable legal requirements. Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in the patents, applications, published applications and other publications that are herein incorporated by reference, the definition set forth in this section prevails over the definition that is incorporated herein by reference.

When introducing elements of the present disclosure or the embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. It is understood that aspects and embodiments of the disclosure described herein include “consisting” and/or “consisting essentially of” aspects and embodiments.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

Various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### Compositions

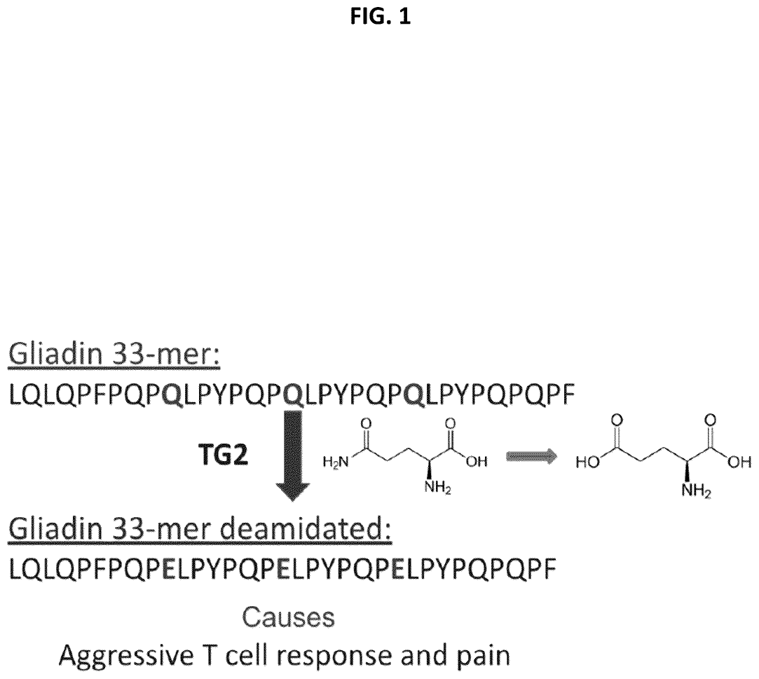

In some embodiments, the present disclosure provides compositions for the breakdown of gluten proteins. In some embodiments, the gluten proteins comprise immunogenic gluten proteins. In some embodiments, the gluten proteins comprise gliadin. Gliadin is a major protein component of gluten, and several gliadin peptides are recognized by T-cells of the CeD patient's intestines. While most gliadin peptides are digested prior to reaching the intestine, 33-mer and 25-mer gliadin peptides often remain undigested. Thus, these two peptides are the primary immunogenic peptides in CeD patient intestines following gluten ingestion. In some embodiments, the gluten proteins comprise 33-mer gliadin peptides. Deamidation of 33-mer gliadin peptides by transglutaminase (TG2) results in a highly immunogenic peptide, FIG. 1, that causes an aggressive T-cell response in the intestines of CeD patients leading to inflammation, pain, malabsorption, fatigues, and a high risk of long-term health complications including malnutrition and cancer. In some embodiments, the gluten proteins comprise 33-mer deaminated gliadin peptides. In some embodiments, the gluten proteins comprise 25-mer gliadin peptides and/or 25-mer gliadin peptides.

In some embodiments, the composition comprises fruit extract-derived enzymes for the breakdown of gluten proteins. In some embodiments, the fruit extract-derived enzymes include one or more of papain, bromelain, and actinidin. Papain is a protease derived from papaya. Bromelain is a protease derived from pineapple. Actinidin is a protease derived from kiwi. In some embodiments, the fruit extract-derived enzymes additionally include proteases derived from figs, guava, or mango, or any other protease-rich fruit. In some embodiments, the composition comprises proteases derived from honey, ginger root, or any other protease derived from a plant or animal source.

In some embodiments, the fruit extract-derived enzymes include papain, bromelain, and actinidin. In some embodiments, the ratio of papain to bromelain to actinidin is 1:2:3 (“1-2-3 EC mix”). In some embodiments, the ratio of papain to bromelain to actinidin is a different ratio, for example, a 1:3:6 ratio, a 1:4:6 ratio, a 2:3:4 ratio, a 2:3:6 ratio, or another ratio.

In some embodiments, the composition additionally comprises at least one digestive enzyme. In some embodiments, the digestive enzyme comprises one or more of protease, pepsin, amylase, lipase, or lactase. Proteases and pepsin break down proteins. Amylases break down complex carbohydrates. Lipases break down fats. Lactases break down lactose. Given the digestion and malabsorption issues faced by many CeD patients, digestive enzymes beyond proteases and pepsin may contribute to reduce gastrointestinal distress and assist in digestion in these patients.

In some embodiments, the composition additionally comprises a probiotic. In some embodiments, the probiotic comprises one or more strains of Lactobacillus or Bifidobacterium. In some embodiments, the probiotic comprises other strains probiotic bacteria. Probiotics may also assist in digestion and reducing gastrointestinal distress in CeD patients, and some strains may contribute to the breakdown of gluten proteins.

Various embodiments of the disclosure have been described herein. It should be recognized that these embodiments are merely illustrative of the present disclosure. Variations of those preferred embodiments may become apparent to those of ordinary skill in the art upon reading the foregoing description. It is expected that skilled artisans can employ such variations as appropriate, and the disclosure is intended to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated or otherwise clearly contradicted by context.

### EXAMPLES

### Example 1: Validation and Testing of Digestive Enzymes for Gluten Breakdown

### Gliadin Peptide Treatments

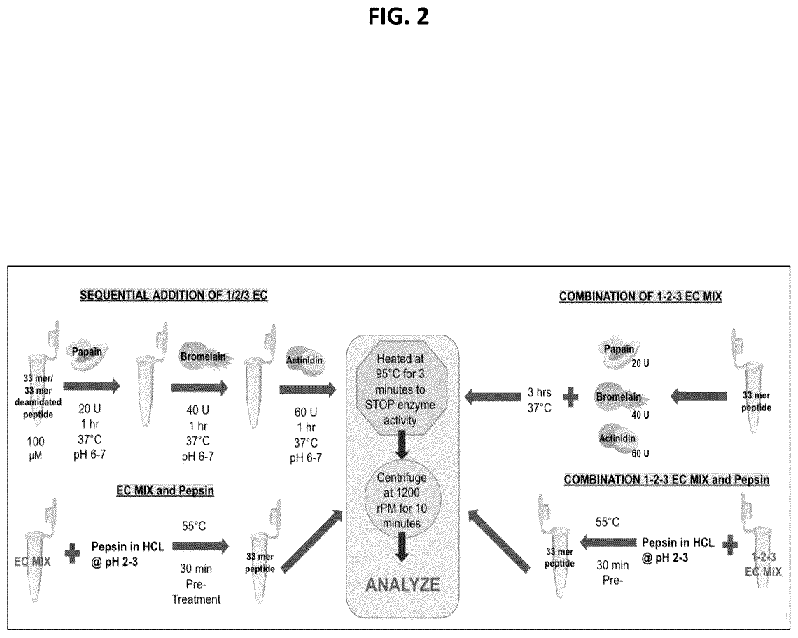

Generally, 100 μM of purified 33-mer gliadin peptide or purified 33-mer deamidated gliadin peptide were treated for three hours at 37° C. with various enzyme mixes. FIG. 2. Enzyme mixes included: (a) papain, bromelain, and actinidin at 20U: 40U: 60U (“EC 1-2-3 mix”), respectively, added for the three hour incubation at pH 6-7; (b) papain, bromelain, and actinidin at 20U: 40U: 60U, respectively, wherein the enzymes were added sequentially each hour (“sequential monotherapy”), starting with papain, at pH 6-7; and (c) EC 1-2-3 mix incubated with 0.5 mg/ml pepsin in HCl at a pH of 2-3 for 30 minutes at 55° C. prior to the three hour incubation with peptide. FIG. 2. Treatment reactions were heated to 95° C. for three minutes to stop enzyme activity, centrifuged at 1200RPM for 10 minutes, and then the supernatant was analyzed.

### LC MS Analysis

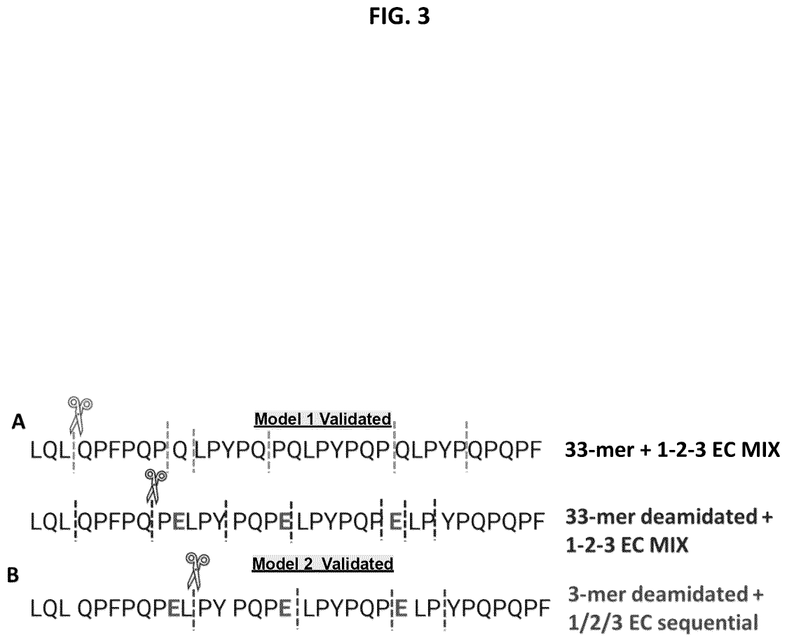

Following enzymatic treatments, samples comprising (1) purified 33-mer gliadin peptide; (2) purified 33-mer deamidated gliadin peptide; (3) mix (a) from above, wherein the peptide was purified 33-mer gliadin peptide; (4) mix (a) from above, wherein the peptide was purified 33-mer deamidated gliadin peptide; (5) mix (b) from above, wherein the peptide was purified 33-mer gliadin peptide; and (6) mix (b) from above, wherein the peptide was purified 33-mer deamidated gliadin peptide, were each mixed with 5% acetonitrile and 0.1% formic acid and analyzed via LC/MS. Both the 1-2-3 EC mix samples (3 and 4) and the sequential monotherapy samples (5 and 6) showed the breakdown of gliadin peptides. FIG. 3.

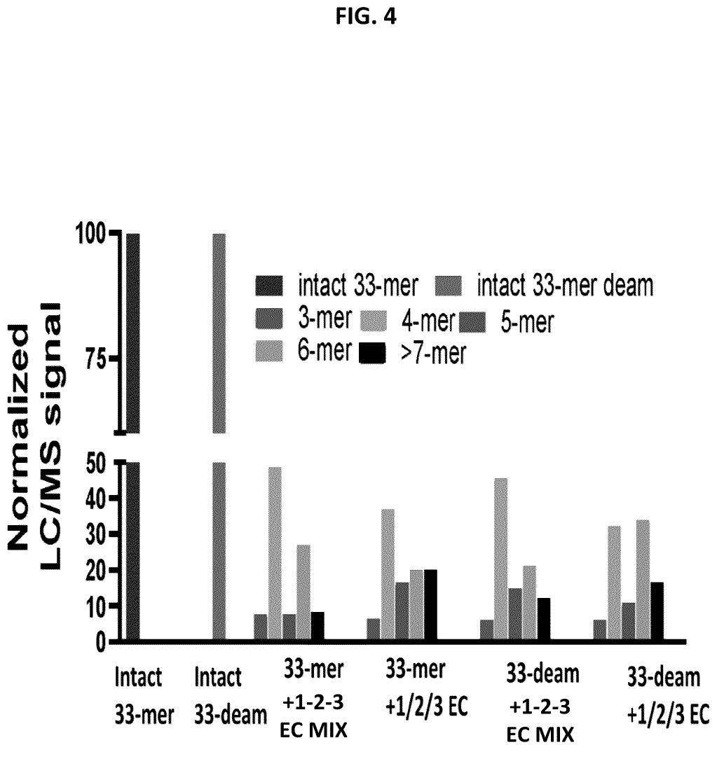

The cleavage sites of the 33-mer gliadin peptides and 33-mer deamidated gliadin peptides were identified for both the 1-2-3 EC mix samples and the sequential monotherapy samples, FIG. 3, and the breakdown amino acid fragments of each sample were quantitated via LC/MS, FIG. 4.

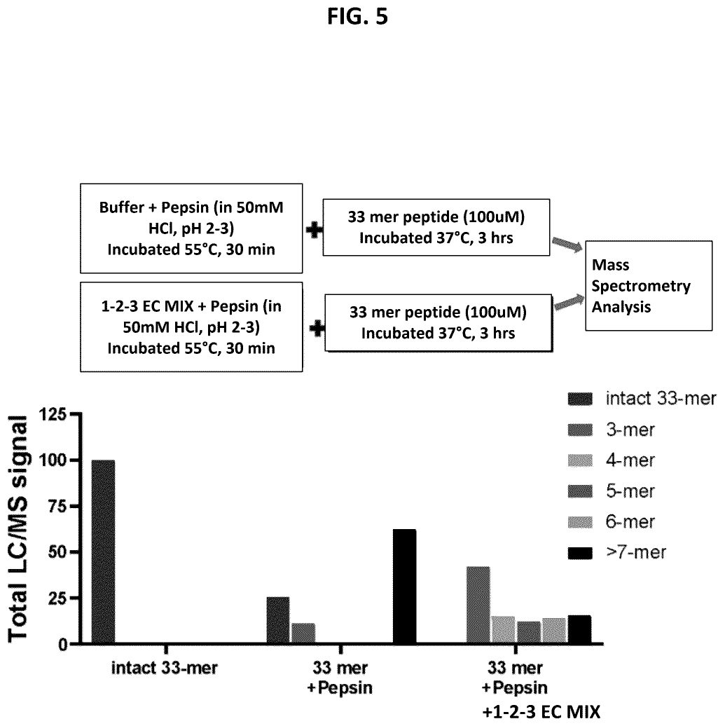

The effectiveness of 1-2-3 EC mix in the stomach acid environment was then analyzed via LC/MS. The breakdown of 33-mer gliadin peptides in the presence of pepsin and HCl was compared to the breakdown of 33-mer gliadin peptides in the presence of enzyme mix (c) above. The presence of pepsin in HCl alone was not enough to effectively break down 33-mer gliadin peptides, while 1-2-3 EC mix in the stomach environment broke the 33-mer gliadin peptide down into multiple smaller subunits. FIG. 5.

### Confirmation of Gliadin Peptide Breakdown

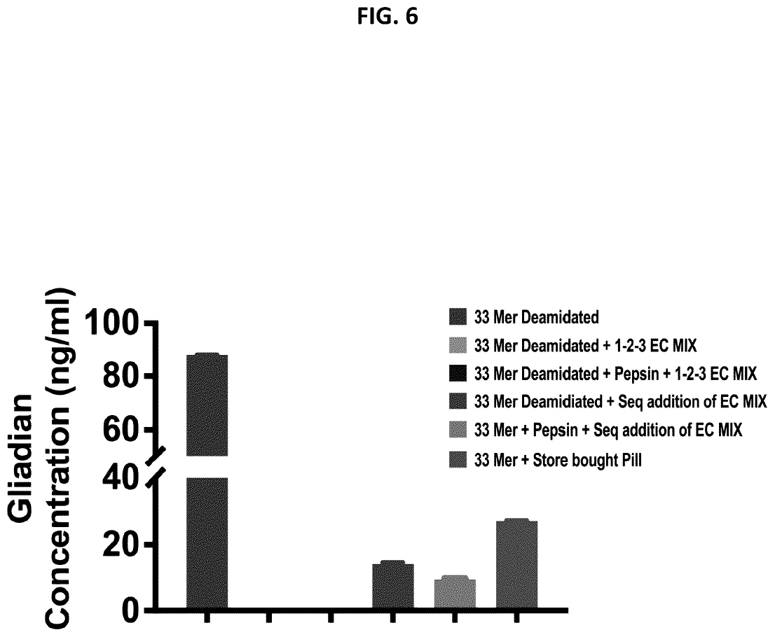

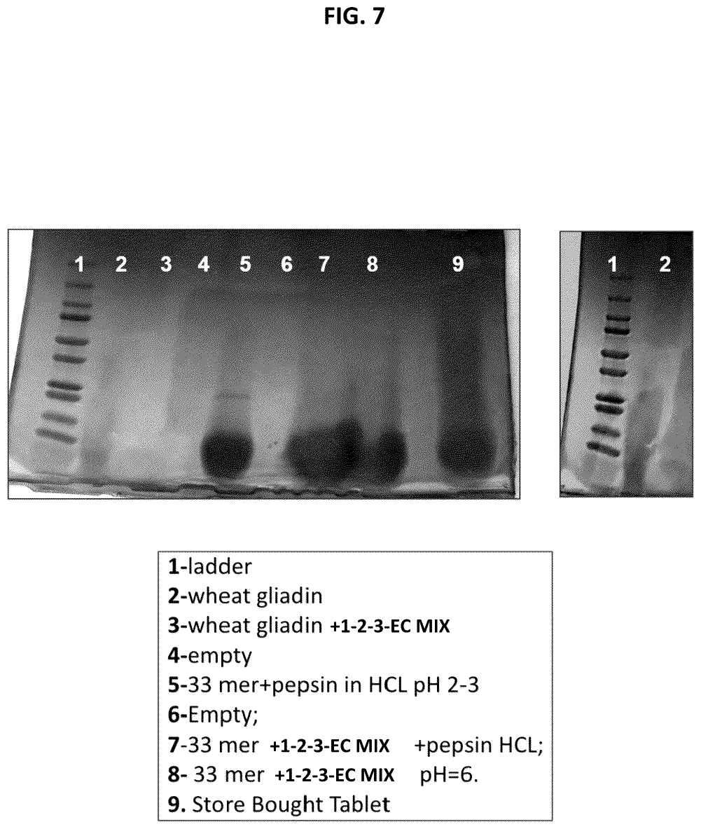

Measurements of 33-mer gliadin peptide concentrations were obtained from samples of gliadin 33-mer peptides and 33-mer deamidated gliadin peptides treated with mixes (a), (b) and (c). 1-2-3 EC mix both at a pH of 6 and in combination with pepsin at a pH of 2 significantly degraded 33-mer deamidated gliadin peptides. FIG. 6. The complete breakdown of 33-mer gliadin peptide following treatment with 1-2-3 EC mix in the presence of pepsin at a pH of 2 was confirmed by SDS-PAGE analysis. FIG. 7.

### Confirmation of Reduced Immunogenicity of Treated Gliadin Peptides

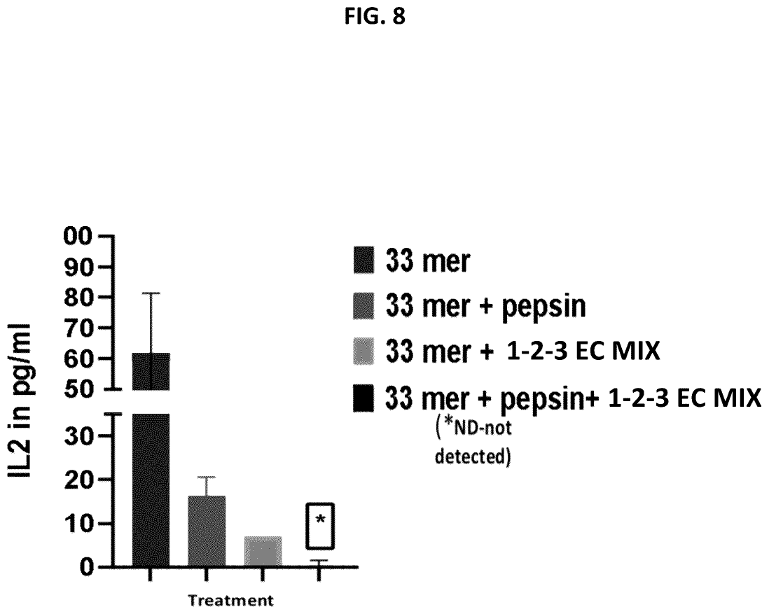

5KC murine T-cells deficient in T-cell receptors transduced with human CD4 T cell receptors from celiac disease patients were obtained from the lab of Dr. Aaron Michels. 33-mer gliadin peptides treated with 1-2-3 EC mix in the presence of pepsin reduced T-cell stimulation, assessed via IL-2 secretion, to undetectable levels. FIG. 8.

While the invention has been described in detail, modifications within the spirit and scope of the invention will be readily apparent to those of skill in the art. It should be understood that aspects of the invention and portions of various embodiments and various features recited above and/or in the appended claims may be combined or interchanged either in whole or in part. In the foregoing descriptions of the various embodiments, those embodiments which refer to another embodiment may be appropriately combined with other embodiments as will be appreciated by one of ordinary skill in the art. Furthermore, those of ordinary skill in the art will appreciate that the foregoing description is by way of example only, and is not intended to limit the invention. All U.S. applications, patents and publications cited herein are incorporated by reference in their entirety.

### REFERENCES

1. Jan Petersen, Jeroen van Bergen, Khai Lee Loh, Yvonne Kooy-Winkelaar, Dennis X. Beringer, Allan Thompson, Sjoerd F. Bakker, Chris J. J. Mulder, Kristin Ladell, James E. McLaren, David A. Price, Jamie Rossjohn, Hugh H. Reid, Frits Koning; Determinants of Gliadin-Specific T Cell Selection in Celiac Disease. J Immunol 15 Jun. 2015; 194 (12): 6112-6122. https://doi.org/10.4049/jimmunol.1500161

2. Ostrov D A, Alkanani A, McDaniel K A, Case S, Baschal E E, Pyle L, Ellis S, Pollinger B, Seidl K J, Shah V N, Garg S K, Atkinson M A, Gottlieb P A, Michels A W. Methyldopa blocks MHC class II binding to disease-specific antigens in autoimmune diabetes. J Clin Invest. 2018 May 1;128 (5): 1888-1902. doi: 10.1172/JC197739. Epub 2018 Apr. 3. PMID: 29438107; PMCID: PMC5919818.

3. Wei G, Helmerhorst E J, Darwish G, Blumenkranz G, Schuppan D. Gluten Degrading Enzymes for Treatment of Celiac Disease. Nutrients. 2020 Jul. 15;12 (7): 2095. doi: 10.3390/nu12072095. PMID: 32679754; PMCID: PMC7400306.dem mass spectrometry. Sci Rep. 2017 Mar. 22;7:45092. doi: 10.1038/srep45

4. Alves T O, D'Almeida C T S, Scherf K A, Ferreira M S L. Modern Approaches in the Identification and Quantification of Immunogenic Peptides in Cereals by LC-MS/MS. Front Plant Sci. 2019 Nov. 14; 10:1470. doi: 10.3389/fpls.2019.01470. PMID: 31798614; PMCID: PMC6868032.092. PMID: 28327674; PMCID: PMC5361186.

5. Qiao S W, Bergseng E, Molberg Ø, Xia J, Fleckenstein B, Khosla C, Sollid L M. Antigen presentation to celiac lesion-derived T cells of a 33-mer gliadin peptide naturally formed by gastrointestinal digestion. J Immunol. 2004 Aug. 1;173 (3): 1757-62. doi: 10.4049/jimmunol.173.3.1757. PMID: 15265905.

6. Characteristics of adult celiac disease in the USA: results of a national survey. Green, P. H. et.al. American Journal of Gastroenterology, 2001, 2006.

7. Green P H, Fleischauer A T, Bhagat G, Goyal R, Jabri B, Neugut A I. Risk of malignancy in patients with celiac disease. Am J Med. 2003 Aug. 15;115 (3): 191-5. doi: 10.1016/s0002-9343 (03) 00302-4. PMID: 12935825.

8 Podkaminer K K, Shao X, Hogsett D A, Lynd L R. Enzyme inactivation by ethanol and development of a kinetic model for thermophilic simultaneous saccharification and fermentation at 50° C. with Thermoanaerobacterium saccharolyticum ALK2. Biotechnol Bioeng. 2011 June; 108 (6): 1268-78.

9. Hochegger R, Mayer W, Prochaska M. Comparison of R5 and G12 Antibody-Based ELISA Used for the Determination of the Gluten Content in Official Food Samples. Foods. 2015;4 (4): 654-664. Published 2015 Nov. 9. doi: 10.3390/foods4040654

10. White J, Pullen A, Choi K, Marrack P, Kappler J W. Antigen recognition properties of mutant V beta 3+T cell receptors are consistent with an immunoglobulin-like structure for the receptor. J Exp Med. 1993 Jan. 1;177 (1): 119-25. doi: 10.1084/jem.177.1.119. PMID: 8380294; PMCID: PMC2190864.

That which is claimed is:

1. A composition for the breakdown of gluten proteins.

2. The composition of claim 1, wherein the gluten proteins comprise gliadin.

3. The composition of claim 2 wherein the gliadin comprises 33-mer gliadin peptides.

4. The composition of claim 2, wherein the gliadin comprises 33-mer gliadin deamidated gliadin peptides.

5. The composition of claim 1, comprising one or more fruit extract-derived enzymes.

6. The composition of claim 5, comprising one or more of papain, bromelain, and actinidin.

7. The composition of claim 6, comprising papain, bromelain, and actinidin.

8. The composition of claim 7, wherein the ratio of papain to bromelain to actinidin is 1:2:3.

9. The composition of claim 5, additionally comprising at least one digestive enzyme.

10. The composition of claim 9, wherein the at least one digestive enzyme comprise one or more of protease, pepsin, amylase, lipase, or lactase.

11. The composition of claim 5, additionally comprising a probiotic.

12. The composition of claim 11, wherein the probiotic comprises one or more strains of Lactobacillus and/or Bifidobacterium.
